(19) United States
(12) Patent Application Publication
(10) Pub. No.: US 2012/0253142 A1
(43) Pub. Date: Oct. 4, 2012

(54) MONITORING, PREDICTING AND TREATING CLINICAL EPISODES

(75) Inventors: Guy Meger, Haifa (IL); Zvi Shinar, Binyamina (IL); Roman Karasik, Lod (IL); Tal Klap, Netanya (IL); Efrat Herbst, Tel Aviv (IL); Avner Halperin, Ramat Gan (IL)

(73) Assignee: EARLYSENSE LTD., Ramat Gan (IL)

(21) Appl. No.: 13/389,200

(22) PCT Filed: Dec. 7, 2011
(86) PCT No.: PCT/IL11/50045

§ 371 Date: Jun. 13, 2012

Related U.S. Application Data
(60) Provisional application No. 61/420,402, filed on Dec. 7, 2010.
(60) Provisional application No. 61/439,971, filed on Feb. 7, 2011.
(60) Provisional application No. 61/561,962, filed on Nov. 21, 2011.

(51) Int. Cl. A61B 5/1455; A61B 5/08; A61B 5/11

(52) U.S. Cl. 600/301

(57) ABSTRACT

Apparatus and methods are described including a motion sensor (30) that senses motion of a subject and generates a motion signal in response thereto. An oximetry sensor (86) measures oxygen saturation of the subject and generates an oximetry signal in response thereto. A control unit (14) analyzes the sensed motion and the sensed oximetry signal, and filters out false alerts relating to a condition of the subject generated by the oximetry signal, based on correlation between the oximetry signal and an aspect of the motion signal. Other embodiments are also described.

### CROSS-REFERENCES TO RELATED APPLICATIONS

The present application claims priority from the following U.S. provisional patent applications, which are incorporated herein by reference:

U.S. 61/420,402 to Meger, filed Dec. 7, 2010;

U.S. 61/439,971 to Meger, filed Feb. 7, 2011; and

U.S. 61/561,962 to Meger, filed Nov. 21, 2011.

### FIELD OF EMBODIMENTS OF THE INVENTION

The present invention relates generally to monitoring patients and predicting and monitoring abnormal physiological conditions and treating those conditions, and specifically to methods and apparatus for predicting and monitoring abnormal physiological conditions by non-contact measurement and analysis of characteristics of physiological and/or physical parameters.

### BACKGROUND

Chronic diseases are often expressed by episodic worsening of clinical symptoms. Preventive treatment of chronic diseases reduces the overall dosage of required medication and associated side effects, and lowers mortality and morbidity. Generally, preventive treatment should be initiated or intensified as soon as the earliest clinical symptoms are detected, in order to prevent progression and worsening of the clinical episode and to stop and reverse the pathophysiological process. Therefore, the ability to accurately monitor pre-episodic indicators increases the effectiveness of preventive treatment of chronic diseases.

Many chronic diseases cause systemic changes in vital signs, such as breathing and heartbeat patterns, through a variety of physiological mechanisms. For example, common respiratory disorders, such as asthma, chronic obstructive pulmonary disease (COPD), sleep apnea and cystic fibrosis (CF), are direct modifiers of breathing and/or heartbeat patterns. Other chronic diseases, such as diabetes, epilepsy, and certain heart conditions (e.g., congestive heart failure (CHF)), are also known to modify cardiac and breathing activity. In the case of certain heart conditions, such modifications typically occur because of pathophysiologies related to fluid retention and general cardiovascular insufficiency. Other signs such as coughing and sleep restlessness are also known to be of importance in some clinical situations.

Many chronic diseases induce systemic effects on vital signs. For example, some chronic diseases interfere with normal breathing and cardiac processes during wakefulness and sleep, causing abnormal breathing and heartbeat patterns.

Breathing and heartbeat patterns may be modified via various direct and indirect physiological mechanisms, resulting in abnormal patterns related to the cause of modification. Some respiratory diseases, such as asthma, and some heart conditions, such as CHF, are direct breathing modifiers. Other metabolic abnormalities, such as hypoglycemia and other neurological pathologies affecting autonomic nervous system activity, are indirect breathing modifiers.

### SUMMARY OF EMBODIMENTS

Embodiments of the present invention provide methods and systems for monitoring patients for the occurrence or recurrence of a physiological event, for example, a chronic illness or ailment. This monitoring assists the patient or healthcare provider in treating the ailment or mitigating the effects of the ailment. Embodiments of the present invention provide techniques for monitoring vital and non-vital signs using automated sensors and electronic signal processing, in order to detect and characterize the onset of a physiological event, and, for some applications, to treat the event, such as with therapy or medication.

There is therefore provided, in accordance with some applications of the present invention, apparatus including:

a motion sensor configured to sense motion of a subject and to generate a motion signal in response thereto;

an oximetry sensor configured to measure oxygen saturation of the subject and to generate an oximetry signal in response thereto; and

a control unit configured to analyze the sensed motion and the sensed oximetry signal, and filter out false alerts relating to a condition of the subject generated by the oximetry signal, based on correlation between the oximetry signal and an aspect of the motion signal.

For some applications, the control unit is configured to filter out the false alerts based on correlation between the oximetry signal and an aspect of the motion signal relating to a respiratory cycle of the subject.

For some applications, the motion sensor is configured to sense motion of the subject without contacting or viewing the subject or clothes the subject is wearing.

For some applications, the motion sensor includes a camera configured to acquire images of the subject, and the control unit is configured to reduce an identifiability of portions of the subject's body in the images, by applying a contour detection algorithm to the images.

There is further provided, in accordance with some applications of the present invention, apparatus including:

one or more monitors configured to sense data relating to a plurality of patients; and

a set of one or more display units, at least one of the display units being configured to:
associate colors with respective groups of the patients, based upon respective caregivers who are assigned to the groups of patients, and
display the sensed data relating to each patient, in the color of the group to which the patient belongs.

For some applications, the apparatus further includes mobile alert devices that are assigned to the caregivers, the mobile alert devices being assigned to respective caregivers, based upon the color of the group of patients to which the caregiver is assigned.

There is additionally provided, in accordance with some applications of the present invention, apparatus for use with a bed that includes an active surface that moves, the apparatus including:

a sensor configured to sense motion of a subject in the bed, and generate a motion signal in response thereto; and

a control unit configured to:
analyze the sensed motion,
determine whether at least a component of the motion signal was generated by movement of the active surface of the bed,
in response thereto, filter the motion signal to remove from the motion signal the component of the motion signal that was generated by movement of the active surface of the bed, and
generate an output in response to the filtered motion signal.

For some applications, the active surface includes an active surface that is powered by an electric power line, and the control unit is configured to remove from the motion signal the component of the motion signal that was generated by movement of the active surface of the bed by removing from the motion signal a component of the signal having a frequency that is characteristic of the electric power line.

For some applications, the control unit is configured to determine whether at least a component of the motion signal was generated by movement of the active surface of the bed, by utilizing a clustering algorithm.

For some applications, the control unit is configured to determine whether at least a component of the motion signal was generated by movement of the active surface of the bed, by determining that a variability of a parameter of the component of the signal is below a threshold variability level.

For some applications, the control unit is configured to determine whether at least a component of the motion signal was generated by movement of the active surface of the bed, by determining that a standard deviation of an amplitude of the component of the signal is below a threshold.

For some applications, the control unit is configured to determine whether at least a component of the motion signal was generated by movement of the active surface of the bed, by determining that a standard deviation of a period of the component of the signal is below a threshold.

There is further provided, in accordance with some applications of the present invention, apparatus including:

a sensor configured to sense motion of a subject in a bed, and generate a motion signal in response thereto; and

a control unit configured to:
determine a level of restlessness of the subject in response to the sensed motion,
in response thereto, generate an alert to a clinician to assign a turn protocol to the subject.

For some applications, the control unit is configured to identify the activation of an active surface by analyzing the motion signal, and the control unit is configured to generate an alert to the clinician to change the subject's turn protocol, in response to the identification of the activation of the active surface.

For some applications, in response to the level of restlessness of the subject, the control unit is configured to indicate the turn protocol that should be assigned to the subject.

For some applications, the control unit is configured to identify the activation of an active surface by analyzing the motion signal, and the control unit is configured to modulate the turn protocol, in response to the identification of the activation of the active surface.

For some applications, the control unit is configured to identify subject turn events by analyzing the sensed motion, and the control unit is configured to generate an alert in response to detecting that the subject has not turned in accordance with the turn protocol.

For some applications, the control unit is configured to identify a subject turn event both in response to a clinician indicating that the subject was turned, and in response to analysis of the sensed motion showing that the subject was turned.

For some applications, the control unit is configured to:

run a countdown timer, the control unit being configured to generate an alert to the clinician to turn the subject, in response to the countdown timer,

detect a posture change of the subject by analyzing the motion signal, and

reset the countdown timer, in response to the detected posture change.

There is additionally provided, in accordance with some applications of the present invention, apparatus including:

a sensor configured to sense motion of a subject in a bed, and generate a motion signal in response thereto; and

a control unit configured to determine from the sensed motion a parameter of the subject selected from the group consisting of: respiration rate, and heartbeat, the control unit including bed-exit detection functionality configured to:
determine a likelihood that the subject will exit the bed within a given time period, by analyzing the sensed motion, the time period being between 30 seconds to 60 minutes,
determine that the likelihood has increased in response to detecting an increase in the selected parameter, and
generate an alert in response to determining that the likelihood is greater than a threshold likelihood.

For some applications, the control unit is configured to modulate the threshold in response to a history of bed exits by the subject.

There is further provided, in accordance with some applications of the present invention, apparatus including:

a sensor configured to sense motion of a subject in a bed, and generate a motion signal in response thereto; and

a control unit configured to determine from the sensed motion a parameter of the subject selected from the group consisting of: respiration rate, and heartbeat, the control unit including bed-exit detection functionality configured to:
determine a likelihood that the subject will exit the bed within a given time period, in response to the sensed motion and in response to detecting an increase in the selected parameter, the time period being between 30 seconds to 60 minutes, and
generate an alert in response to determining that the likelihood is greater than a threshold likelihood.

There is additionally provided, in accordance with some applications of the present invention, apparatus including:

a sensor configured to sense motion of a subject in a bed, and generate a motion signal in response thereto; and

a control unit configured to receive an input from the subject that generates a call to a nurse, the control unit including bed-exit detection functionality configured to:
determine a likelihood that the subject will exit the bed within a given time period, in response to the sensed motion and in response to receiving the input from the subject, the time period being between 30 seconds to 60 minutes, and
generate an alert in response to determining that the likelihood is greater than a threshold likelihood.

For some applications, the control unit is configured to modulate the threshold in response to a history of bed exits by the subject.

There is further provided, in accordance with some applications of the present invention, apparatus including:

a sensor configured to sense motion of a subject in a bed, and generate a motion signal in response thereto; and

a control unit including bed-exit detection functionality configured to:
determine a likelihood that the subject will exit the bed within a given time period, in response to the sensed motion and in response to a history of bed exits by the subject, the time period being between 30 seconds to 60 minutes, and
generate an alert in response to determining that the likelihood is greater than a threshold likelihood.

For some applications, the control unit is configured to receive an input from the subject that generates a call to a nurse, and the control unit is configured to modulate the threshold in response to receiving the input from the subject.

For some applications, the control unit is configured to determine from the sensed motion a parameter selected from the group consisting of: a respiration rate of the subject and a heartbeat of the subject, and modulate the threshold in response to one or more of the selected parameters.

There is further provided, in accordance with some applications of the present invention, apparatus including:

a first sensor configured to detect temperature of a subject;

a second sensor configured to detect a non-temperature parameter of the subject; and

a control unit configured to identify that the subject has undergone a temperature change, in response to the temperature detected by the first sensor and the parameter detected by the second sensor.

For some applications, the second sensor is configured to detect a heart rate of the subject.

For some applications, the second sensor includes a contact-less sensor that is configured to detect the non-temperature parameter, without contacting or viewing the subject or clothes that the subject is wearing.

For some applications, the first sensor includes a contact-less sensor that is configured to detect the subject's temperature without contacting or viewing the subject or clothes that the subject is wearing.

There is additionally provided, in accordance with some applications of the present invention, apparatus for use with a bed, a top section of which can be tilted, the apparatus including:

a motion sensor configured to sense motion of a subject in the bed, and to generate a motion signal in response thereto;

a sensor configured to sense a tilt angle of the top section of the bed; and

a control unit configured to:
detect a presence of the subject in the bed in response to the motion signal, and
generate an alert in response to detecting that, while the subject in the bed, the tilt angle is less than a threshold tilt angle for greater than a threshold time period.

There is further provided, in accordance with some applications of the present invention, apparatus for use with an artificially ventilated subject lying on a bed, a top section of which bed can be tilted, the apparatus including:

a first sensor configured to detect that the subject is being ventilated and to generate a ventilation-indication signal in response thereto;

a second sensor configured to sense a tilt angle of the top section of the bed; and

a control unit configured to analyze the ventilation-indication signal and the tilt angle and to generate an alert in response thereto.

For some applications, the control unit is configured to generate the alert in response to detecting that, while the subject is being ventilated, the tilt angle is less than a threshold tilt angle for greater than a threshold time period.

For some applications, the first and second sensors include contact-less sensors that are configured, respectively, to detect the ventilation signal and the tilt angle without contacting or viewing the subject or clothes that the subject is wearing.

For some applications, the first sensor is further configured to detect respiratory motion of the subject, and the control unit is configured to generate an output in response to the detected respiratory motion and the detected ventilation signal.

There is additionally provided, in accordance with some applications of the present invention, apparatus including:

a respiratory sensor configured to sense respiratory motion of a subject in a bed, and to generate a respiratory signal in response thereto;

an oximetry sensor configured to detect oxygen saturation of a subject, and to generate an oxygenation signal in response thereto; and

a control unit configured to:
determine a correlation between the respiratory signal and the oxygenation signal, and
generate an alert that is indicative of an abnormal respiratory condition of the subject, in response to detecting a change in the correlation.

For some applications, the control unit is configured to generate the alert in response to detecting a decrease in the correlation between the respiratory signal and the oxygenation signal.

For some applications, the respiratory sensor includes a contact-less sensor that is configured to detect the subject's respiratory motion without contacting or viewing the subject or clothes that the subject is wearing.

For some applications, the control unit is configured to generate the alert in response to:

determining that the subject has stopped breathing, in response to the respiratory signal, and

determining that, since the subject stopped breathing, the time it takes the oxygen saturation level to drop by a threshold amount is less than a threshold time period.

There is further provided, in accordance with some applications of the present invention, apparatus for monitoring a subject, including:

a motion sensor configured to detect motion of the subject and to generate a motion signal in response thereto; and

a control unit configured to:
determine that the subject has not turned in accordance with a turn protocol of the subject in response to the motion signal,
determine whether the subject is at a given stage of a sleep cycle of the subject, and
generate an alert indicating that the subject should be turned in response to determining that (a) the subject has not been turned in accordance with the subject's turn protocol, and (b) the subject is at the given stage of the subject's sleep cycle.

For some applications, the control unit is configured to detect activation of an active surface by analyzing the motion signal, and modulate the turn protocol of the subject in response to identifying the activation of the active surface.

For some applications, the control unit is configured to identify the activation of an active surface by analyzing the motion signal, and the control unit is configured to generate an alert to a clinician to change the subject's turn protocol, in response to the identification of the activation of the active surface.

There is additionally provided, in accordance with some applications of the present invention, apparatus for monitoring a subject, including:

a sensor configured to detect a physiological parameter of the subject and to generate a signal in response thereto; and

a control unit including:
a pattern analysis module configured to analyze the signal generated by the sensor; and
a sound generation module configured, in response to the analysis of the signal by the pattern analysis module, to generate an audio output that is based upon a sound template that mimics a sound related to the physiological parameter.

For some applications, the sensor is configured to detect respiration of the subject, and the sound generation module is configured to generate the audio output by generating an audio output that is based upon a sound template that mimics a sound of respiration selected from the group consisting of: an inspiration sound and an expiration sound.

For some applications, the sound generation module includes a template module configured to generate the sound template.

For some applications, the sound generation module is configured to generate the audio output by generating an audio output that is based upon a synthetic sound template that mimics the sound related to the physiological parameter.

For some applications, the control unit is configured to receive an input that is indicative of a parameter of the subject selected from the group consisting of: an age of the subject, a gender of the subject, and a physical state of the subject, and the sound generation module is configured to generate the audio output by modulating the sound template, in response to the input to the control unit.

For some applications, the pattern analysis module is configured to determine a characteristic of the physiological parameter by analyzing the signal, and the sound generation module is configured to generate the audio output by modulating the sound template, responsively to the determined characteristic of the physiological parameter.

There is further provided, in accordance with some applications of the present invention, apparatus for use with a patient who shares a bed with a second person, including:

a motion sensor configured to detect motion of the patient and the second person and to generate a motion signal in response thereto; and

a control unit including a patient identification module configured to identify components of the motion signal that were generated by the patient, by distinguishing between components of the motion signal that were generated respectively by the patient and by the second person,

the control unit being configured to analyze the components of the motion signal that were generated by the patient and to generate an output in response thereto.

For some applications, the patient identification module is configured to identify components of the motion signal that were generated by the patient, by identifying components of the motion signal that have a signal strength that is a characteristic signal strength of a motion signal of the patient.

For some applications, the patient identification module is configured to identify components of the motion signal that were generated by the patient by identifying components of the motion signal that have a pattern that is a characteristic pattern of motion of the patient.

For some applications, the patient identification module includes a weight sensor that is configured to detect when the patient is lying above the motion sensor.

For some applications, the motion sensor is configured to facilitate the identification of components of the motion signal that were generated by the patient, by strengthening a signal strength of the components of the motion signal that are generated by the patient.

For some applications, the apparatus is for use with a patient who lies on a mattress, and the sensor is configured to be placed at a position selected from the group consisting of: underneath the mattress at a position that is higher than a head of the patient is typically placed, and adjacent to and in contact with a side of the mattress.

For some applications, the sensor is configured such as to facilitate identification, by the patient identification module, of components of the motion signal that were generated by a longitudinal cardio-ballistic effect of the patient.

There is additionally provided, in accordance with some applications of the present invention, apparatus for use with a subject lying on a mattress on a bed, the apparatus including:

a motion sensor configured to sense motion generated by a longitudinal cardio-ballistic effect of the subject, by at least partially being placed adjacent to and in contact with a side of the mattress, and not under the mattress; and

a support element configured to maintain the contact between the motion sensor and the side of the mattress.

For some applications, the support element includes an element disposed at a right angle with respect to the sensor, the support element being configured to be placed underneath the mattress.

For some applications, the support element includes a stretchable band that is configured to be placed around sides of the mattress by being stretched, the band being configured to maintain the contact between the motion sensor and the side of the mattress by shrinking.

For some applications, the side of the mattress is for placement next to a surface, and the support element includes a compressible member configured to be placed between the mattress and the surface adjacent to the mattress, the support element being configured to maintain the contact between the motion sensor and the side of the mattress by expanding against the side of the mattress.

The present invention will be more fully understood from the following detailed description of embodiments thereof, taken together with the drawings, in which:

### BRIEF DESCRIPTION OF THE DRAWINGS

FIG. 1 is a schematic illustration of a system for monitoring a chronic medical condition of a subject, in accordance with some applications of the present invention;

FIGS. 2A-C are schematic block diagrams illustrating components of a control unit of the system of FIG. 1, in accordance with some applications of the present invention;

FIGS. 2D-E are schematic illustrations of a sensor, in accordance with some applications of the present invention;

FIG. 3 is a schematic block diagram illustrating a breathing pattern analysis module of the control unit of FIG. 2A, in accordance with some applications of the present invention;

FIG. 4 is a graph showing a motion signal measured on an active surface, in accordance with some applications of the present invention;

FIG. 5 is a graph showing a motion signal of a subject, in accordance with some applications of the present invention;

FIGS. 6A-B show cluster analysis results of a respiratory related motion signal, measured in accordance with some applications of the present invention;

FIGS. 7A-B are graphs showing a motion signal measured on a subject lying on an active surface, in accordance with some applications of the present invention;

FIGS. 8A-B are graphs showing signals that were generated simultaneously by, respectively, a motion sensor and an oximetry sensor, in accordance with some applications of the present invention; and

FIGS. 9A-B are graphs showing enlargements of portions of the signals shown, respectively, in FIG. 8A and FIG. 8B, the signals being generated in accordance with some applications of the invention.

### DETAILED DESCRIPTION OF EMBODIMENTS

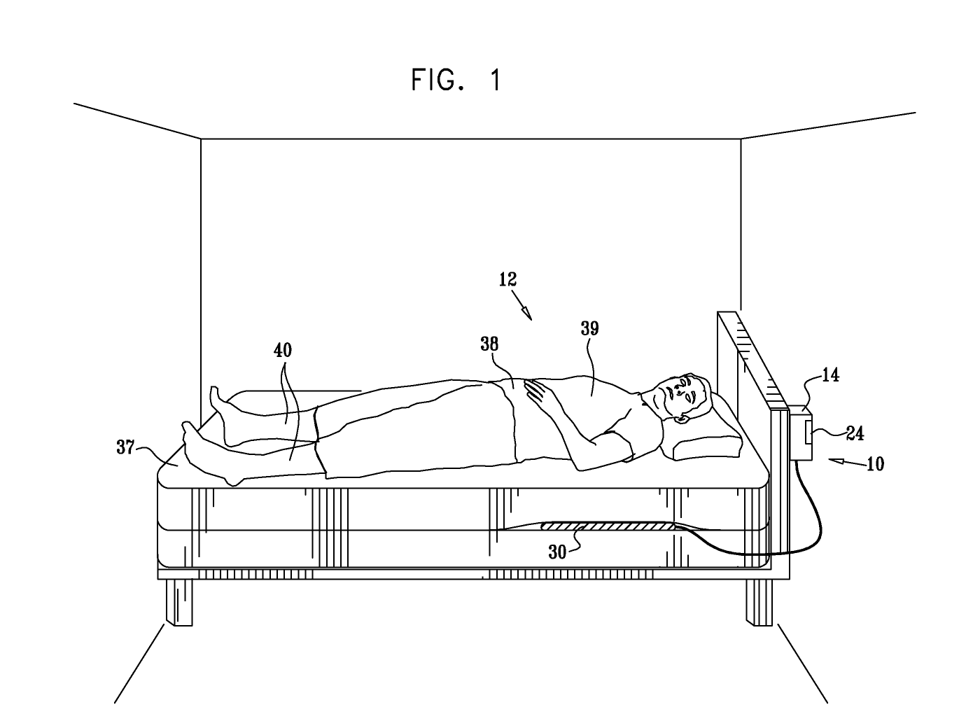

FIG. 1 is a schematic illustration of a system 10 for monitoring a chronic medical condition of a subject 12, in accordance with some applications of the present invention. System 10 typically comprises a motion sensor 30, a control unit 14, and a user interface (U/I) 24. System 10 is generally similar to system 10 described in WO 09/138,976 to Meger, which is incorporated herein by reference, except for differences described herein. For some applications, user interface 24 is integrated into control unit 14, as shown in the figure, while for other applications, the user interface and the control unit are separate units. For some applications, motion sensor 30 is integrated into control unit 14, in which case user interface 24 is either also integrated into control unit 14 or remote from control unit 14.

In some embodiments of the present invention, motion sensor 30 is a “non-contact sensor,” that is, a sensor that does not contact the body of subject 12 or clothes subject 12 is wearing. In other embodiments, motion sensor 30 does contact the body of subject 12 or clothes subject 12 is wearing. In the former embodiments, because motion sensor 30 does not come in contact with subject 12, motion sensor 30 detects motion of subject 12 without discomforting or inconveniencing subject 12. For some applications, motion sensor 30 performs sensing without the knowledge of subject 12, and even, for some applications, without the consent of subject 12. For some applications, motion sensor 30 does not have a direct line of sight with subject 12 or the clothes subject 12 is wearing.

Motion sensor 30 may comprise a ceramic piezoelectric sensor, vibration sensor, pressure sensor, or strain sensor, for example, a strain gauge, configured to be installed under a reclining surface 37, and to sense motion of subject 12. The motion of subject 12 sensed by sensor 30, during sleep, for example, may include regular breathing movement, heartbeat-related movement, and other, unrelated body movements, as discussed below, or combinations thereof. For some applications, sensor 30 comprises a standard communication interface (e.g. USB), which enables connection to standard monitoring equipment.

For some applications, in addition to wirelessly-enabled motion sensor 30, control unit 14 is coupled to one or more sensors 60 applied to subject 12, such as a blood oxygen monitor 86 (e.g., a pulse oximeter/photoplethysmograph), an ECG monitor 62, or a temperature sensor 80. In accordance with respective applications, one or more of sensors 60 is a contact sensor or a contact-less sensor.

Most of the experimental results presented in the present application were measured using one or more piezoelectric sensors. Nevertheless, the scope of the present invention includes performing measurements with other motion sensors 30, such as other pressure gauges or accelerometers.

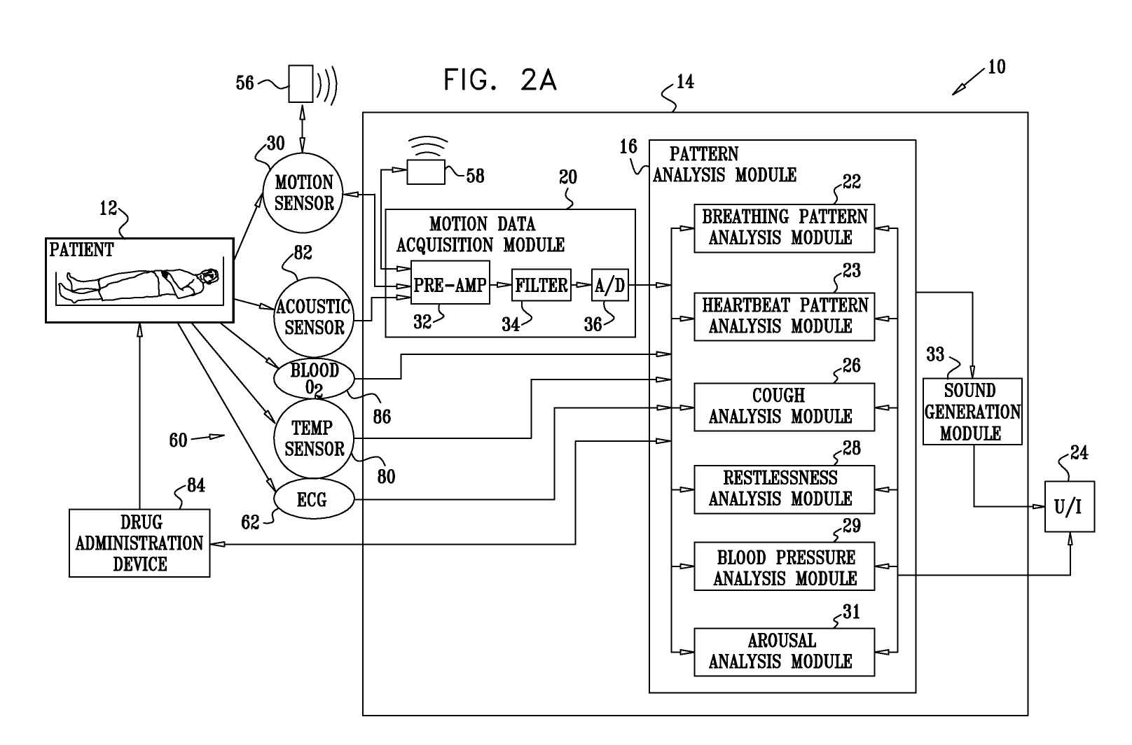

FIG. 2A is a schematic block diagram illustrating components of control unit 14 in accordance with some applications of the present invention. Control unit 14 typically comprises a motion data acquisition module 20 and a pattern analysis module 16. Pattern analysis module 16 typically comprises one or more of the following modules: a breathing pattern analysis module 22, a heartbeat pattern analysis module 23, a cough analysis module 26, a restlessness analysis module 28, a blood pressure analysis module 29, and an arousal analysis module 31. For some applications, two or more of analysis modules 20, 22, 23, 26, 28, 29, and 31 are packaged in a single housing. For other applications, the modules are packaged separately (for example, so as to enable remote analysis, by one or more of the pattern analysis modules, of breathing signals acquired locally by data acquisition module 20).

User interface 24 typically comprises a dedicated display unit, such as an LCD or CRT monitor. Alternatively or additionally, the user interface 24 comprises a wireless or wired communication port for relaying the acquired raw data and/or processed data to a remote site for further analysis, interpretation, expert review, and/or clinical follow-up. For example, the data may be transferred over a telephone line, and/or over the Internet or another wide-area network, either wirelessly or via wires.

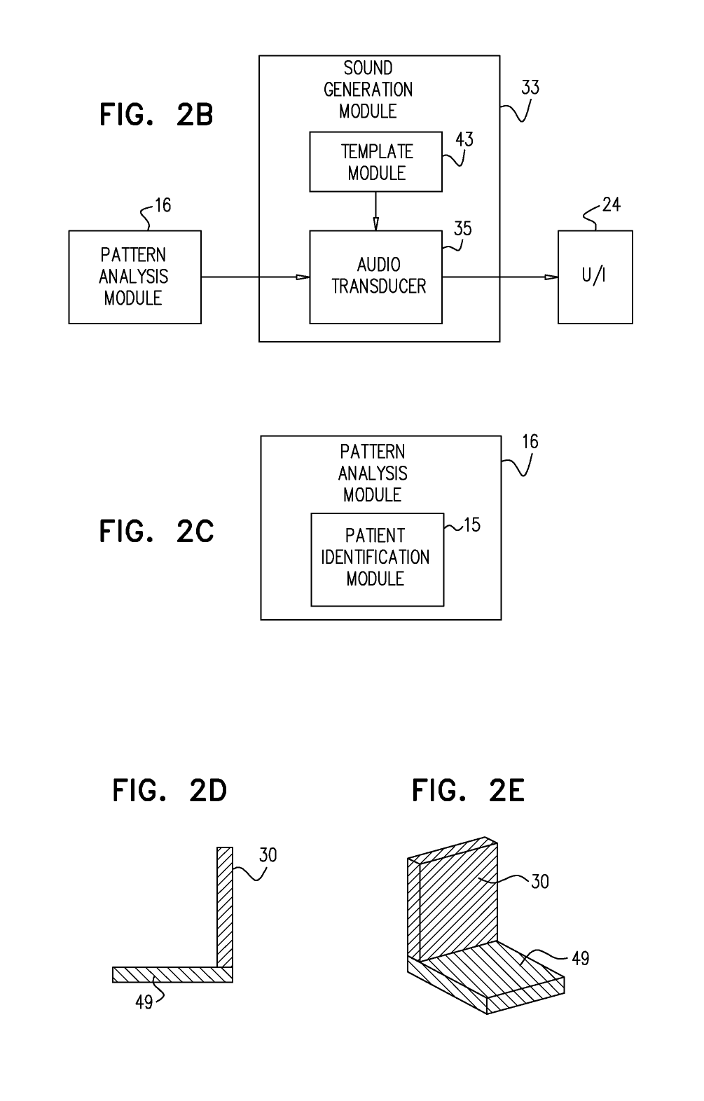

Breathing pattern analysis module 22 is configured to extract breathing patterns from the motion data, as described hereinbelow with reference to FIG. 3, and heartbeat pattern analysis module 23 is configured to extract heartbeat patterns from the motion data. Alternatively or additionally, system 10 comprises another type of sensor, such as an acoustic or air-flow sensor attached or directed at the subject's face, neck, chest, and/or back, or placed under the mattress.

In some applications of the present invention, system 10 comprises a temperature sensor 80 for measurement of body temperature. For some applications, temperature sensor 80 comprises an integrated infrared sensor for measurement of body temperature. Body temperature is a vital sign indicative of general status of systemic infection and inflammation. Global rise in body temperature is used as a first screening tool in medical diagnostics.

For some applications, the control unit includes a sound generation module 33. FIG. 2B is a schematic block diagram of components of the sound generation module, in accordance with some applications of the present invention. As shown, the sound generation module typically includes an audio transducer 35, and a template module 43.

For some applications, a real time indication of the heart and/or the respiratory rate of the subject, and/or movement of the subject, is provided to the clinician, in a non-visual manner, e.g., in audio format. For example, in an operating room (e.g., during the performance of a gastrointestinal or plastic surgery procedure), a surgeon and/or anesthesiologist may find it useful to have provided to him/her an indication of each heartbeat, breath and/or movement of the subject in a non-visual manner, e.g., in audio format. Thus, the clinician is able to sense changes in heart or respiratory rates or patterns, and/or movements of the subject without having to look at a visual display.

For some applications, sound generation module 33 is configured to translate a motion signal into sound. Pattern analysis module 16 identifies features of respiration (e.g., inhalation, exhalation, and/or apnea), features of movement, and/or phases of the subject's heartbeat, based on data sensed by one or more of the sensors shown in FIG. 2A, and/or based on data sensed by a plethysmograph, a respiratory inductive sensor, and/or a piezoelectric belt.

Template module 43 generates at least one sound template (e.g., synthetic or recorded sound templates) that mimics a sound of respiration, movement and/or heartbeat. It is noted that the template module typically generates sound templates that sound similar to sounds of the physiological parameter that the sound templates represent, rather than generating tones, or beeps that are representative of, but do not sound similar to, sounds of the physiological parameter. Thus, in applications in which the sound generation module is configured to generate a sound template that is representative of the patient's respiration, the template module generates sound templates that sound like sounds of a person's respiration cycle (such as inspiration and expiration). In applications in which the sound generation module is configured to generate a sound template that is representative of the patient's heartbeat, the template module generates sound templates that sound like sounds of a heart beating. The inventors hypothesize that hearing sounds that mimic sounds of the physiological parameter that the sound templates represent provides a more intuitive feedback to a clinician who is operating on the subject, than tones or beeps that do not sound like the physiological parameter itself. Furthermore, clinicians who have been told about the aforementioned sound generation module, and who have been shown a demonstration thereof, have confirmed it to be the case that hearing sounds that mimic sounds of the physiological parameter that the sound templates provide a more intuitive feedback to a clinician.

Typically, the duration and/or amplitude of the sound templates are modulated such as to fit with the features identified by pattern analysis module 14. Typically, respective templates are generated by the template module depending on the subject's age, gender, and/or physical state, one or more of the aforementioned parameters typically being provided as an input to the system. For some applications, the pitch, amplitude, and/or other characteristics of the templates are adapted in accordance with parameters of the monitored signal. For example, the amplitude of the audio signal may be modulated in response to the amplitude of the breathing motion signal, and/or the pitch of the audio signal may be modulated in response to the period of the respiration and/or cardiac cycle. The modulated templates are typically formatted as an audio file, and audio transducer 35 plays the file (at normal, high, or low speed), e.g., via user interface 24. In accordance with respective applications, the audio file is played in real time, or at a delay with respect to the signals that were generated by the subject.

FIG. 2C is a schematic block diagram of pattern analysis module 16 of control unit 14 of system 10, in accordance with some applications of the present invention. For some applications, the pattern analysis module includes a patient identification module 15. The patient identification module is configured to determine which motion signals detected by motion sensor 30 were generated by the patient. For example, in cases in which the patient who is being monitored is sharing a bed with a second person (e.g., the patient's wife), the patient identification module determines which components of the motion signal detected by the motion sensor were generated by the patient and which were generated by the second person. The pattern analysis module then analyzes the components of the signal that were generated by the patient, and generates outputs (such as alerts), as described herein, in response thereto. For some applications, the patient identification module is configured to determine when the patient is out of bed by determining that the motion signal detected by the motion detector is being generated by the second person. For some applications, the patient identification module is configured to determine which components of the motion signal detected by the motion sensor were generated by the patient even when the patient is smaller than the second person.

For some applications, patient identification module 15 is configured to determine which components of the motion signal detected by motion sensor 30 were generated by the patient using one or more of the following techniques:

a. The patient identification module identifies patterns (e.g., a respiratory pattern, a heart rate pattern, and/or a motion pattern) that are characteristic of, respectively, the patient and the second person. The patient identification module then determines that components of the signal that correspond to the characteristic patterns of the patient have been generated by the patient. For some applications, the patient identification module learns characteristic patterns of the patient by utilizing a weight sensor (e.g., as described hereinbelow), and/or or utilizing long term average patterns of the patient. For some applications, in response to an input to system 10, the pattern identification module operates in a learning mode, in which the module learns characteristic patterns of the patient.

b. The patient identification module identifies characteristic signal strengths generated, respectively, by the patient and by the second person. For example, the sensor may be disposed underneath the patient who lies on a first side of the bed and the second person may typically lie on the second side of the bed. In such cases, signals generated by the patient are typically characterized as being of greater strength than those generated by the second person. Alternatively, the patient may be smaller than the second person, and may therefore generate signals that are characterized as being weaker than signals generated by the second person.

Reference is now made to FIGS. 2D-E, which are schematic illustrations of respective views of motion sensor 30, in accordance with some applications of the present invention. For some applications, motion sensor 30 is configured to facilitate determination by the patient identification module 15 of which components of the motion signal were generated by the patient. For example, the sensor may be placed in a position, and/or shaped, such as to strengthen the signal that is received from the patient. For some applications, the sensor is placed underneath the patient's mattress at a position higher than where the patient rests his/her head, such that the strongest signals that the sensor receives are those generated by the longitudinal cardio-ballistic effect of the patient. Alternatively or additionally, at least a portion of the sensor is placed adjacent to and in contact with a side of the patient's mattress (e.g., the head of the patient's mattress), and not underneath the mattress. For some applications, the motion sensor comprises at least a portion of an L-shaped structure, as shown in FIGS. 2D-E. The structure is shaped to define horizontal and vertical portions that form approximately (or precisely) a right angle with one another. The horizontal portion of the structure is placed underneath the patient's mattress, and the vertical portion of the structure is placed adjacent to and in contact with a side of the patient's mattress (e.g., the head of the patient's mattress). For some applications, the horizontal portion of the structure does not perform any sensing functionalities but acts as a support element 49 for supporting the vertical portion adjacent to and in contact with a side of the patient's mattress (e.g., the head of the patient's mattress), the vertical portion acting as sensor 30.

Alternatively or additionally, a different support element is used to support sensor 30 at a position adjacent to and in contact with a side of the patient's mattress (e.g., the head of the patient's mattress). For example, a compressible member (such as a cushion) may be placed between the side of the mattress and a surface (e.g., a wall or a headboard) that is adjacent to the side of the mattress, and may be configured to hold the sensor against the head of the mattress, by expanding against the side of the mattress. For some applications, the sensor is disposed on a stretchable band (e.g., an elastic band). The band is stretched in order to facilitate placement of the band around the sides of the patient's mattress, and the band then shrinks, such as to maintain the sensor adjacent to and in contact with a side of the patient's mattress (e.g., the head of the patient's mattress). For some applications, the sensor is not disposed on a stretchable band, but the sensor is maintained adjacent to and in contact with a side of the patient's mattress (e.g., the head of the patient's mattress), using a stretchable band.

For some applications, the motion sensor includes a weight sensor that is configured to measure a weight that is placed on top of the weight sensor, and to identify that the patient is lying above the motion sensor in response thereto. The patient identification module identifies signals from the motion sensor as having been generated by the patient, in response to the signal generated by the weight sensor. For some applications, the weight sensor is used to determine when the subject is directly on top of the weight sensor. In response to determining that the subject is directly on top of the weight sensor, the pattern identification module operates in a learning mode, in which the module learns characteristic patterns of the patient, as described hereinabove. For some applications, respective first and second motion sensors are placed underneath the patient and the second person who uses the bed. Patient identification module 15 determines which components of the motion signal were generated by the patient in response to the signals from both the first and the second motion sensors.

FIG. 3 is a schematic block diagram illustrating components of breathing pattern analysis module 22, in accordance with some applications of the present invention. System 10 is generally similar to breathing pattern analysis module 22, described in WO 09/138,976 to Meger, which is incorporated herein by reference, except for differences described herein. For example, for some applications, breathing pattern analysis module is used with control unit 14 shown in FIG. 2A of the present application. Breathing pattern analysis module 22 analyzes changes in breathing patterns, typically during sleep. Breathing pattern analysis module 22 typically comprises a digital signal processor (DSP) 41, a dual port RAM (DPR) 42, an EEPROM 44, and an I/O port 46. Modules 23, 26, 28, 29, and 31 may be similar to module 22 shown in FIG. 3. For example, modules 23, 26, 28, 29, and 31 may include a digital signal processor, a dual port RAM, an EEPROM, and an I/O port similar to digital signal processor 41, dual port RAM 42, EEPROM 44, and I/O port 46.

In some applications of the present invention, data acquisition module 20 is configured to non-invasively monitor breathing and heartbeat patterns of subject 12. Breathing pattern analysis module 22 and heartbeat pattern analysis module 23 are configured to extract breathing patterns and heartbeat patterns respectively from the raw data generated by data acquisition module 20, and to perform processing and classification of the breathing patterns and the heartbeat patterns, respectively.

Breathing pattern analysis module 22 and heartbeat pattern analysis module 23 are configured to analyze the respective patterns in order to (a) predict an approaching clinical episode, such as an asthma attack, heart condition-related lung fluid buildup, sepsis, cardiac arrest, or respiratory depression, and/or (b) monitor the severity and progression of a clinical episode as it occurs. User interface 24 is configured to notify subject 12 and/or a healthcare worker of the predicted or occurring episode. Prediction of an approaching clinical episode facilitates early preventive treatment, which generally improves outcomes, e.g., by lowering required dosages of medication, and/or lowering mortality and morbidity. When treating a hospitalized patient in a general care ward, for example, an earlier identification of patient deterioration may prevent the need to admit the patient to the ICU, shorten his length of stay, and increase the likelihood for successful recovery to discharge.

Normal breathing patterns in sleep are likely to be subject to slow changes over days, weeks, months and years. Some changes are periodic due to periodic environmental changes, such as a change in seasons, or to a periodic schedule such as a weekly schedule (for example outdoor play every Saturday), or biological cycles such as the menstrual cycle. Other changes are monotonically progressive, for example, changes that occur as children grow or adults age. In some embodiments of the present invention, system 10 tracks these slow changes dynamically.

In some applications of the present invention, system 10 is configured to monitor clinical parameters of the subject including, but not limited to, breathing rate; heart rate; coughing counts; expiration/inspiration ratios; amplitude, number, or frequency of augmented breaths; amplitude, number, or frequency of deep inspirations; amplitude, duration, or frequency of tremors, duration or frequency of sleep cycles, and amplitude, number, or frequency of restlessness patterns. These parameters are examples of “clinical parameters,” as used in the specification and in the claims. In general, a clinical parameter is a numerical parameter that can be measured in a clinical setting and that has clinical value.

Reference is again made to FIG. 1. In some applications of the present invention, motion sensor 30 comprises a pressure/vibration sensor (for example, a piezoelectric sensor) or an accelerometer, which is typically configured to be installed in, on, or under surface 37 upon which the subject lies, e.g., sleeps, and to sense breathing- and heartbeat-related motion of the subject. Typically, surface 37 comprises a mattress, a mattress covering, a sheet, a mattress pad, and/or a mattress cover. For some applications, motion sensor 30 is integrated into surface 37, e.g., into a mattress, and the motion sensor and reclining surface are provided together as an integrated unit. For some applications, motion sensor 30 is configured to be installed in, on, or under surface 37 in a vicinity of an abdomen 38 or chest 39 of subject 12. Alternatively or additionally, motion sensor 30 is installed in, on, or under surface 37 in a vicinity of a portion of subject 12 anatomically below a waist of the subject, such as in a vicinity of legs 40 of the subject. For some applications, such positioning provides a clearer pulse signal than positioning the sensor in a vicinity of abdomen 38 or chest 39 of the subject.

In some applications of the present invention, sensor 30 comprises a single piezoelectric ceramic sensor. The sensor is attached to a plate, e.g., a semi-rigid plate comprising flexible plastic (e.g. Perspex (PMMA), polycarbonate, or acrylonitrile butadiene styrene (ABS)) or non-plastics (e.g., cardboard), for example having dimensions of 20 cm×28 cm×1.5 mm. The sensor is able to detect a signal when the subject assumes most common bed postures, even when the subject's body is not directly above the sensor. In some applications, sensor 30 is implemented using two or more thin piezo-electric sensors (e.g. radius of 13 mm and thickness of 100 um), wherein the two or more sensors are stacked on top of the semi-rigid plate so that the first sensor is attached to the plate and the second (and potentially third, etc.) is attached to the first sensor. The signals from both sensors are added to each other by amplification and/or digitizing electronics, in order to increase the signal to noise ratio of the system.

For some applications, motion sensor 30 (for example, comprising a piezoelectric sensor) is encapsulated in a rigid compartment, which typically has a surface area of at least 10 cm², and a thickness of less than 5 mm. The sensor output is channeled to an electronic amplifier, such as a charge amplifier typically used with piezoelectric sensors, and capacitive transducers to condition the extremely high output impedance of the amplifier to a low impedance voltage suitable for transmission over long cables. The sensor and electronic amplifier translate the mechanical vibrations into electrical signals.

In some applications of the present invention, motion sensor 30 comprises a grid of multiple sensors, configured to be installed in, on, or under reclining surface 37. The use of such a grid, rather than a single unit, may improve breathing and heartbeat signal reception.

In some applications, system 10 includes a posture change identification algorithm that identifies whether a patient has changed his position on a bed or other reclining surface or chair, e.g., using techniques described in WO 09/138,976 to Meger, which is incorporated herein by reference. The objective is to identify whether the patient moved between 1 of the 4 positions: supine, on stomach, on left side, or on right side, since such a change every 2-4 hours is generally required in order to prevent pressure ulcer formation in high risk patients. Alternatively, the system may identify a major body movement that includes a repositioning of the torso and/or the sacrum area that is most prone to pressure ulcer development. The system identifies events of large body motion and evaluates whether they involved a posture change of the main body.

In some applications, a clinician can activate a turn protocol on system 10, whereby the system will remind the clinician to perform a patient turn (i.e., to cause the patient to undergo a posture change) every predetermined threshold period of time. The threshold period of time is typically more than two hours and/or less than four hours, e.g., between two and four hours. System 10 displays a counter of time since the last time a clinician turned the patient and, when the aforementioned threshold period of time has passed since the last time that the clinician turned the patient, the system alerts the clinician to turn the patient. For some applications, the clinician indicates that the patient was turned by means of an input device. Alternatively or additionally, system 10 verifies that a turn was performed, using the motion sensor and/or a camera, e.g., a video camera. For some applications, each performance of a patient turn by the clinician is logged, by the clinician indicating that the turn was performed via the input device, and additionally, an indication of whether the clinician's indicated turn was verified by one or more sensors of system 10 is logged. For some applications, in response to an indication that a turn was performed being logged via the input device, and the indication not having been verified by the system, the system generates an alert. In addition, in some embodiments, if the clinician has activated a turn protocol and the system has detected a patient's posture change without an indication being received from the clinician that the patient was turned by a clinician, the system identifies that as an autonomous turn that was performed by the patient. In such a case, the system may indicate that information to the clinician for him to consider whether there is a need to turn the patient at the next scheduled time and/or to re-evaluate (for example, using standard scales such as Norton or Braden) whether the patient needs to be maintained on a turn protocol. For some applications, this may prevent a clinician from needing to turn a patient unnecessarily, which can be heavily labor intensive.

For some applications, the system is configured to identify an overall level of restlessness that is higher or lower than a threshold value, and/or that is higher or lower than a previous value of restlessness determined for the subject. In response thereto, the system is configured to generate an output that is indicative of a need for the clinician to generate a subject turn protocol, reevaluate an existing turn protocol, and/or reevaluate the Braden score and/or the Norton score of the patient. For some applications, in response to the overall level of restlessness and/or a different parameter of the subject, the system is configured to automatically generate a turn protocol for the subject, and/or adjust an existing turn protocol.

In some applications, system 10 is utilized to reduce patient falls by driving the output unit to generate an alert when a subject sits up in bed, thus providing an early warning for the clinical team for a patient who may be leaving bed to enable assisting him before he actually leaves bed and thus prevent the falls effectively. For some applications, system 10 identifies that the patient has sat up in bed in response to ongoing calculation of the noise level in the motion signal, e.g., as described in WO 09/138,976 to Meger, which is incorporated herein by reference.

For some applications, system 10 includes bed-exit detection functionality that is configured to determine the likelihood of a patient getting out of bed within a given time period (e.g., a time period of between 30 seconds and 60 minutes), in order to provide an early warning indication. For some applications, this may prevent falls that are due to healthcare professionals not being able to respond quickly enough to an alert that is issued in response to detecting that the patient has sat up, or has actually exited his/her bed.

Clinical studies have shown that a patient's getting out of bed is very frequently correlated with the need to go to the bathroom. For some applications, system 10 detects parameters that indicate that patient 12 may be getting out of bed in order to alert and prevent an unescorted bed exit, by detecting parameters that indicate that the patient may need to go to the bathroom, or by detecting other parameters that are indicative of an imminent bed exit by the patient.

For some applications, at least one of the following parameters is detected by the system:
a patient not having been out of bed for a period of time higher than a threshold,
a patient showing a higher restlessness level than a threshold or a previous baseline,
a patient sitting up in bed,
a patient not having been visited by a nurse for a specific period of time,
the time of day, with respect to a patient's sleep cycle, and/or
the time of day
amount of time since patient was under anesthesia or since patient got out of surgery.

System 10 typically detects a likelihood of imminent bed exit at least in part responsively to one or more of the above parameters. For example, system 10 may have a plurality of sensitivity levels for detecting the patient exiting the bed or getting ready to exit the bed. System 10 changes the sensitivity level based, for example, on the amount of time since the patient has last gotten out of bed, or was last visited by a nurse. Thus, for example, if the patient has not been out of bed for a given period of time, e.g., for over three hours (or a different period of time, e.g., five hours, or eight hours), the bed exit sensitivity level is automatically increased to a higher level. Alternatively or additionally, if on the previous day the patient got up at a given time (e.g., 5:00 AM), or if on the previous three days the patient got up at approximately the same time (e.g., at around 5:00 AM), then the bed exit sensitivity level is automatically increased prior to that time (e.g., 15 minutes prior to the time, at 4:45 AM). Further alternatively or additionally, if the nurse has not logged that he/she has visited the patient's room for a given period of time (e.g., for more than two hours) the bed exit sensitivity level is automatically increased. In some applications, if one or more of the above listed criteria is true the system alerts the caregiver that the probability of a bed exit is high. For some applications, the system learns the motion patterns and vital sign patterns prior to a patient getting out of bed from previous days and interprets similar patterns as being indicative of an impending bed exit.

For some applications, system 10 includes motion sensor 30 and also includes an interface to receive as an input the activation of a nurse call. When the nurse call is activated, the bed exit sensitivity level is automatically increased (i.e., the motion threshold in response to which a bed-exit alert is generated is modulated). In many cases, prior to a patient exiting a bed, the patient undergoes an increase in heart rate and/or respiratory rate. Therefore, for some applications, the bed exit sensitivity level is modulated in response to the detected heart rate and/or respiratory rate of the patient. For example, in response to the system detecting an increase in the patient's heart rate of more than 5 bpm (e.g., 5-15 bpm), and/or an increase in respiratory rate of more than 2 breaths/min (e.g., 2-10 breaths/min) the bed exit sensitivity level of the system is increased.

For some applications, system 10 is utilized to detect the change in the body temperature of patient 12, for example, by using a contact-less heat flow sensor such as that described in US 2010/0215074 to Lozinski et al., which is incorporated herein by reference, or by using alternative temperature sensors. For some applications, the objective of such a system is to detect the patient's body temperature, and generate an alert in response to temperature changes in the patient's body, while reducing the generation of false alerts.

In some implementations, the use of the above mentioned heat flow sensor or alternative temperature sensors placed under the sheet or mattress of a bed on which the patient is lying may produce false temperature change readings. For example, a patient's change in posture may affect the heat flow detected by the sensor, and therefore provide a false alert of a change in temperature. For some applications of the present invention, in order to reduce the number of false alerts, the reading of the heat flow sensor is correlated with the reading of a motion, position and/or heart rate sensor. For example, a sensor under the mattress or a camera that detects the patient's posture change is used to detect whether the patient changed position in correlation with the temperature change detected by the heat flow sensor. If no posture change is detected, then the detected temperature change is communicated to the caregiver, and/or an alert is generated. If a change of posture is detected, then, in at least some cases, the system interprets the change in posture as having caused the detected temperature change, and the detected temperature change is filtered out.

Alternatively or additionally, a sensor, such as the motion sensor under the patient's mattress, is used to continuously monitor the patient's heart rate. If the change in heat flow reading correlates with a change in heart rate in the same direction, this is interpreted by the system as indicating that the patient has undergone a temperature change, and the temperature change is communicated to the caregiver, and/or an alert is generated. Otherwise, the detected temperature change is filtered out. For some applications, instantaneous readings of the temperature sensor (e.g., the heat flow sensor) and/or heart rate sensor are used to determine whether the patient has undergone temperature changes, as described. Alternatively, the temperature sensor (e.g., the heat flow sensor), and/or heart rate sensor readings are averaged over a time period in the range of 30 seconds to 60 minutes, in order to determine whether the patient has undergone temperature changes, thereby reducing the generation of false alerts that may result from instantaneous changes in the subject's temperature and/or heart rate.

For some applications, a change in temperature (e.g., a change in heat flow), if correlated with a subject's motion or detected posture change, is used by the control unit as an indication that the posture of the subject has changed. For some applications, a significant drop in the heat flow, if correlated with a subject's motion, is used by the control unit as an indication that the subject has left the bed, and is used in a decision block process (e.g., as described herein) to determine whether the subject has exited the bed.

In some applications, system 10 is connected to a smart bed system with an active surface such as the InTouch Critical Care Bed with an XPRT enabled active surface made by Stryker Medical of Kalamazoo, Mich. The bed is motorized and is able to provide, for example, the following interventions: change the backrest angle, rotate the patient, and/or provide vibration and percussion treatment. System 10 activates one of these interventions in response to the clinical parameters measured. For example, if an increase in the average respiratory rate over a period of 5 minutes to 3 hours (for example 30 minutes) is identified without a corresponding increase in the subject's activity level, which may indicate a deterioration of a patient's respiratory condition, the vibration and percussion treatment is activated or the backrest angle is increased to 30 degrees. Alternatively, if the subject's number of posture changes per time has been below a threshold for a period of time between 1 hour and 24 hours (for example 3 hours), the active surface rotates the patient. Without sensing the subject's rotation, the bed would have to turn the subject every 3 hours, even if he turned autonomously, thus potentially creating a significant and/or unnecessary discomfort to the subject.

In some applications, system 10 is designed to detect the activation of an active surface, via the motion sensor. For some applications, detecting the activation of the active surface allows system 10 to filter out any artifacts that may cause wrong vital sign readings, due to the signal generated by the active surface. Alternatively or additionally, this facilitates the documentation and assessment of the clinical team's compliance with patient care protocols, for example, pressure ulcer prevention protocols.

In order to detect the activation of the active surface, several algorithms are used independently or in combination, for example, as described below:

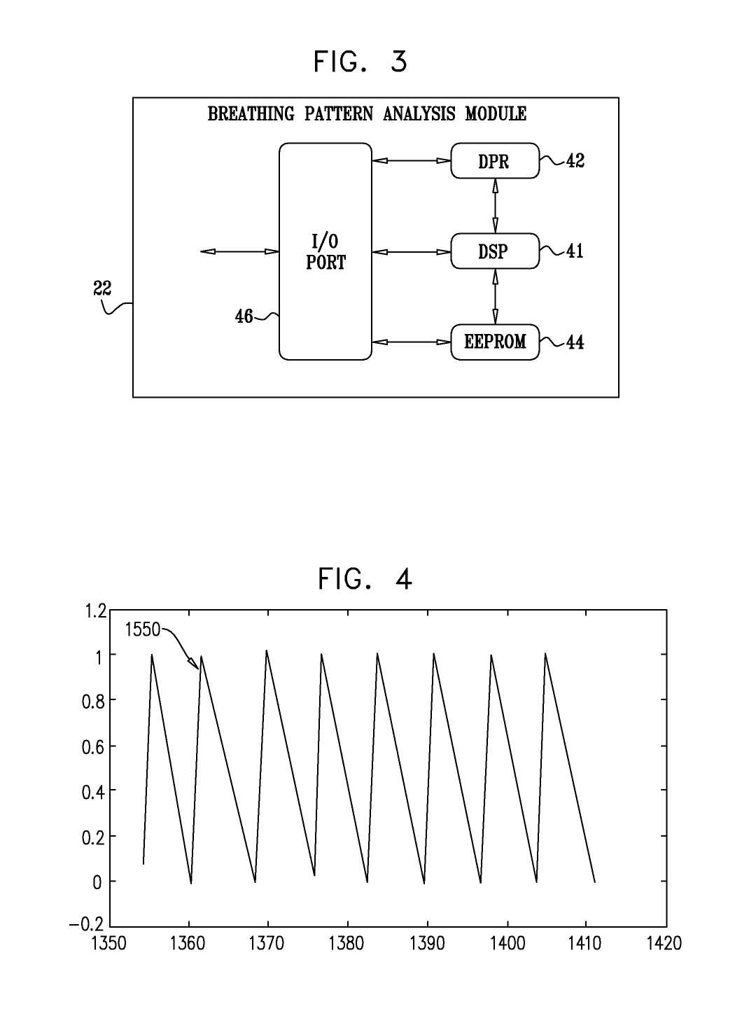

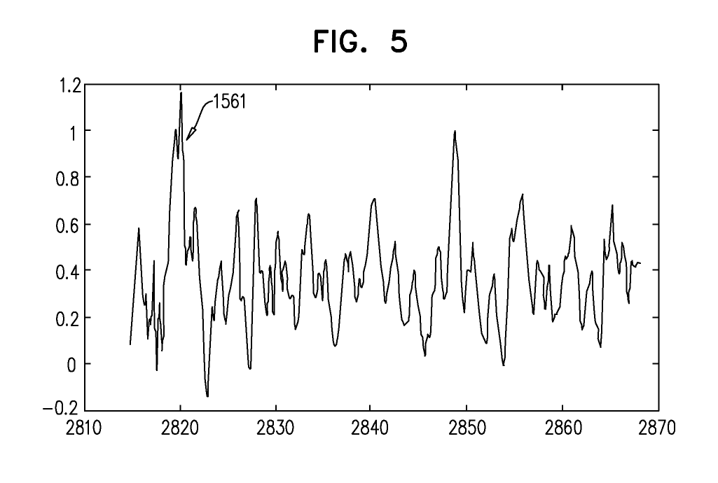

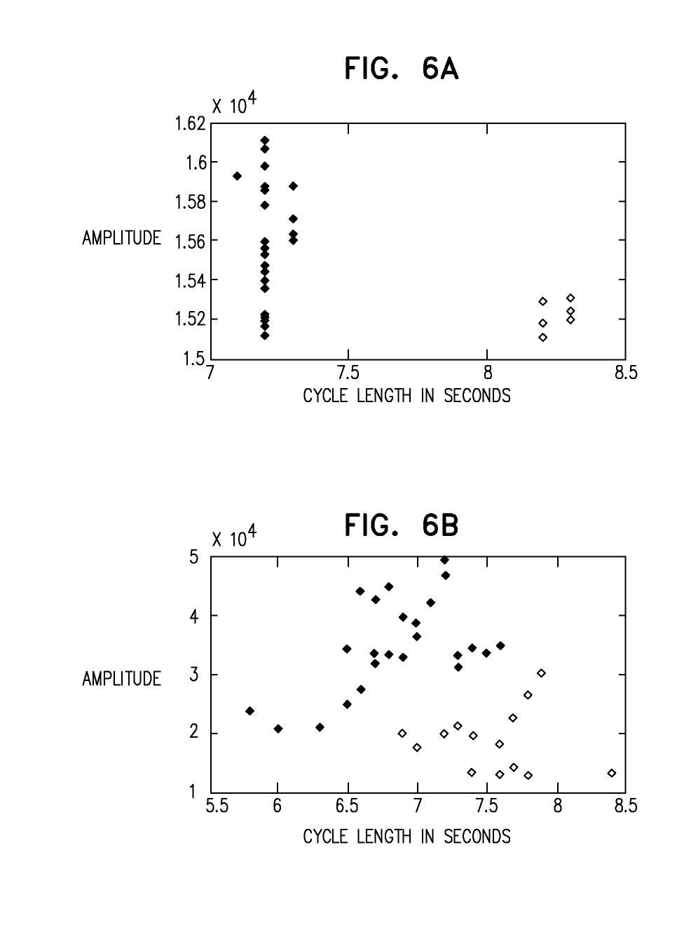

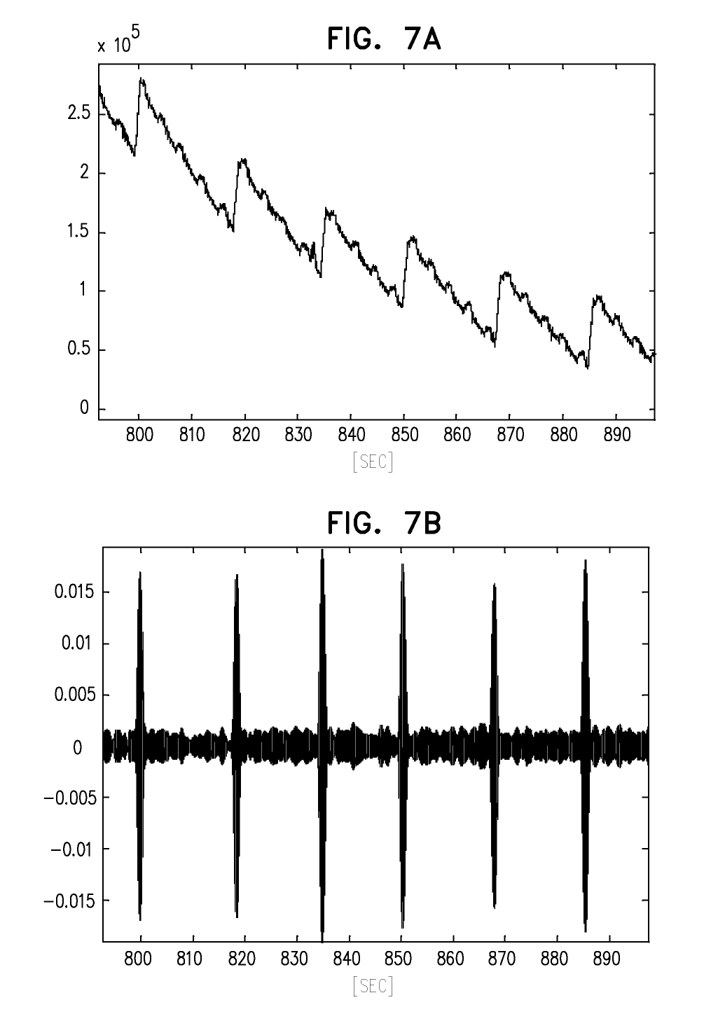

1. Identifying the activation of an electric motor connected to the bed by the detection of the signal having a frequency that is the frequency at which electricity is provided to the system (e.g., 50 or 60 Hz), by utilizing a bandpass filter to filter the mechanical signal. FIG. 7A shows the signal detected from a subject lying on an active surface, the signal being measured in arbitrary units, in accordance with some applications of the present invention. FIG. 7B shows the signal, the signal having been filtered by a 60 Hz bandpass filter. The peaks in FIG. 7B represent the time periods when an active surface is actually activated. In many cases this activation has a characteristic time frequency that is identified by system 10. The detection of activation of a motor on the surface (which, for some applications, is detected by detecting a signal at a given frequency, as described), allows system 10 in some applications, to learn the pattern of the active surface, identify and log its activation, as described below, and/or to filter out any artifacts to the vital sign readings generated by the active surface.
Identifying the mechanical vibration or pressure signal generated by the active surface on the motion sensor as the active surface moves (e.g., by inflating and deflating). Several active surfaces generate a characteristic shape signal on the motion sensor that can be preprogrammed or learned by the system. For example, one such characteristic signal shape is the triangle signal shown in line 1550 of FIG. 4, which shows the shape of the signal of an active surface in arbitrary units, measured using techniques as described herein. Line 1561 of FIG. 5 shows a motion signal in arbitrary units measured on a subject lying on a non-active surface, measured using techniques as described herein. When a patient lies on an active surface a motion signal is generated that is a combination of the motion signal generated by the patient's motion and the motion signal generated by the active surface.
   For some applications, identification of a triangular signal shape such as line 1550 of FIG. 4 is performed in accordance with the technique described below.
   The following criterion for similarity is used:

$$SF = \frac{{\sum }_{k = 1}^{N} A [ k ]}{N} ; A [ k ] = \{ \begin{matrix}1 for ( \vert Sig [ k ] - Triangle [ k ] \vert \leq 0.25 ) \\ 0 for ( \vert Sig [ k ] - Triangle [ k ] \vert > 0.25 )\end{matrix} \qquad ( Equation 1 )$$

Where:
   SF is the similarity factor (this factor is calculated separately for any preprogrammed pattern, such as a triangle pattern);
   Sig is a normalized vector signal derived from the motion sensor;
   Triangle is a normalized pre known triangle shape vector;
   k is the index of the sample in the vector; and
   N is the amount of points compared—length of the above vectors.
If SF is larger than a threshold, for example 0.80, an active surface is identified as being in use by system 10, and the portion of the signal having the triangular shape is not considered to have been caused by the patient's motion.
2. In some applications, system 10 automatically identifies and learns such triangle or other shape patterns generated by the active surface, or other machinery placed on the bed (e.g. a deep vein thrombosis related system). System 10 identifies the pattern and differentiates it from a normal respiratory pattern based on the detection of a repetitive signal that has a very low variation level. For example, if a clustering algorithm is used to detect respiratory motion as described in the section below relating to clustering, triangular or other shaped patterns generated by the active surface will generate dense respiration rate clusters, in terms of cycle length and amplitude of the respiration cycle. Sample criteria for a dense cluster may be, for example: amplitude standard deviation lower than 10% of the average signal amplitude, and/or cycle time (i.e., period) standard deviation lower than 10% of the average cycle time. Typically, a minimum of 6 points are required to be in the cluster. FIG. 6A, which shows clusters that are generated by an active surface, shows data points which have low standard deviation of amplitude results (amplitude being measured in arbitrary units), which are lower than the criterion described above. FIG. 6B, which shows clusters that are generated by human respiratory signal, shows data points having standard deviation of amplitude results (amplitude being measured in arbitrary units) that are higher than the criterion described above.
   An additional example of a technique for detecting a repetitive signal that is indicative of the signal having been generated by an active surface, is by cutting the signal into time segments that are equal to the cycle time of the external signal (i.e., the signal generated by the active surface). These time slots are averaged over a period of time of several cycles, for example, 10 cycles. The resulting average is added to the library of reference signals and used in a similar way to the triangle signal identification described above. For this method, the dominant cycle time is extracted from the spectrum of the signal.

In some applications, system 10 has an input means to receive an indication from the clinician whether the patient 12 is a pressure ulcer risk and is on a patient turn protocol, in order to prevent pressure ulcers. In addition, system 10 has an input or detection means to identify whether the patient is placed on an active surface. System 10 then alerts a clinician, and optionally generates an alert to a supervisor when the turning protocol for such a patient is not followed. For example, in many institutions, any patient who is placed on an active surface should be turned once every two hours. Thus, if system 10 identifies that a patient has been placed on an active mattress, but the turn protocol reminder has not been turned on, and/or the patient is not being turned every 2 hours, the system alerts a clinician. Conversely, if the patient is on a turn protocol but an active surface has not been turned on the system may also alert the clinician. This is useful in ensuring that the full protocol of pressure ulcer prevention is maintained, including both the patient turn aspects, and the active surface utilization.

For some applications, a camera (e.g., a video camera) is used to sense patient motion, e.g., in conjunction with one or more of the motion detection techniques described herein. For some applications, pictures (e.g., still images, or image frames of a video stream) that are detected by the camera are passed through a contour detection algorithm, such as to generate images that contain sufficient data to provide motion detection and analysis, while maintaining the privacy of the patient by reducing the identifiability of the patient and/or portions of the patient's body.

For some applications, the system logs a patient's turns, in addition to analyzing the subject's sleep pattern. In response to both the patient's turn signal and the analysis of the patient's sleep pattern, the system generates an alert to indicate that the patient should be turned. For example, it is typically inconvenient for the patient to be awakened from deep sleep in order to be turned. Therefore, for some applications, the system generates a turn alert in response to the patient's turn signal indicating that the patient needs to be turned, and the patient's sleep pattern analysis indicating that the patient is either awake or at the end of REM sleep stage.

In some cases, a pulse oximeter (e.g., sensor 86) may give erroneous readings without any visible warning. This may happen, for example, because of poor perfusion. In some applications of the present invention, system 10 comprises a pulse oximeter and a motion sensor. System 10 calculates the subject's heart rate using both the pulse oximeter signal and the motion sensor's signal. The system compares the two calculated heart rates to verify that the measured heart rate is correct. If there is a mismatch, the system alerts a healthcare worker.

In some applications, system 10 utilizes the combination of the oximeter and the motion sensor to reduce false alerts. In most cases a significant change in oxygen saturation is expected to be accompanied by a significant change (e.g., an increase or a decrease) in the patient's respiratory rate and/or heart rate, as measured by the motion sensor. To reduce number of false alerts in measuring both oxygen saturation and heart rate via the oximeter sensor, correlation with the data from the motion sensor is used. In some applications, the signal detected by the oximeter sensor is correlated with the respiratory motion signal component detected by the motion sensor. In normal operation of the oximeter, the dominant signal should be correlated with the heart rate related signal and not the respiratory motion signal. However, if the dominant element is correlated with the respiratory signal, the system identifies that the oximeter data is erroneous and the readings and or alerts generated in that channel are filtered out.

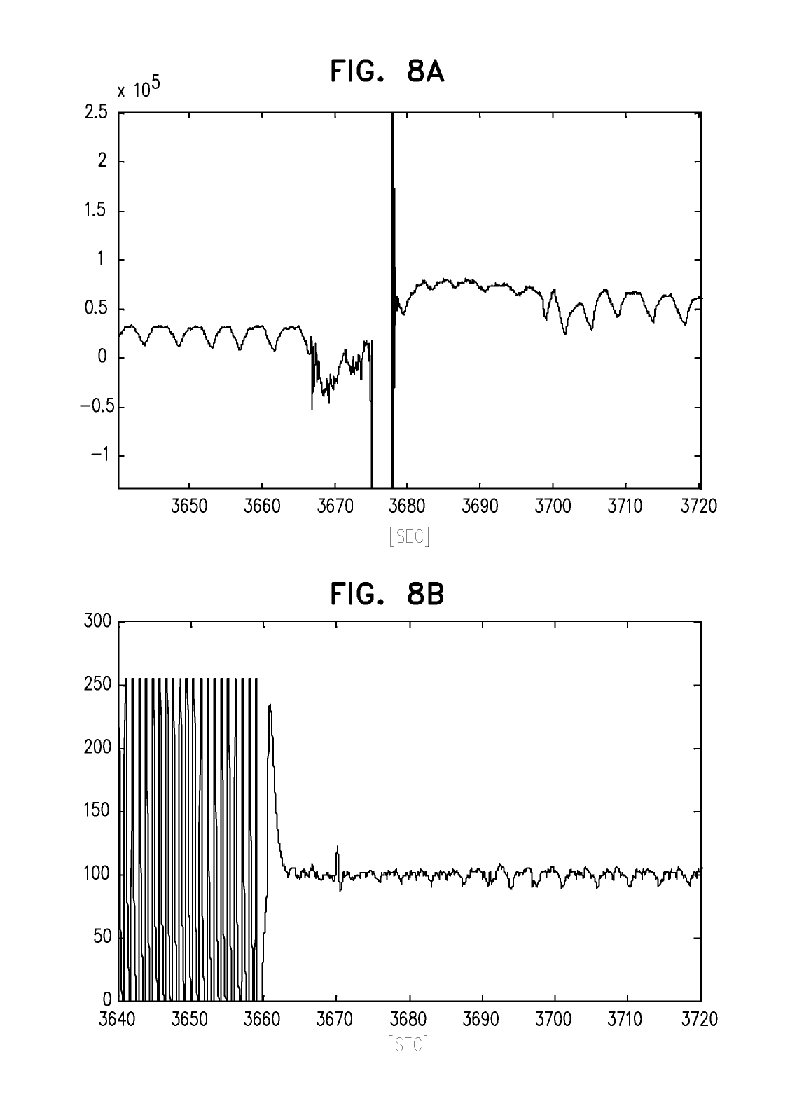

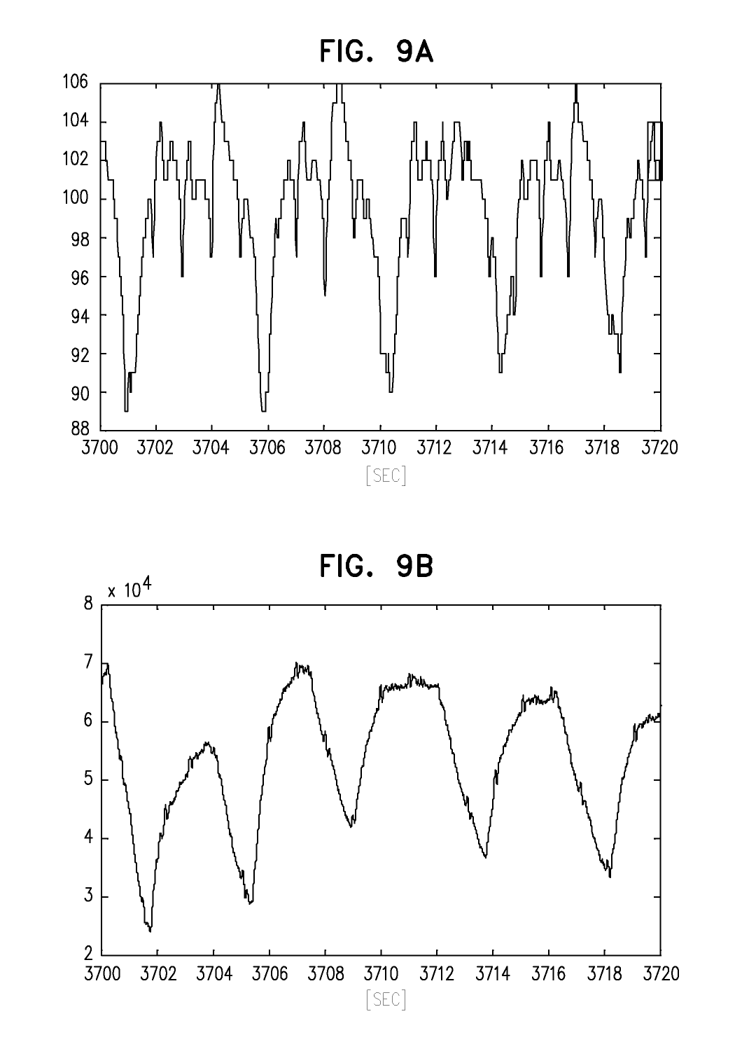

FIGS. 8A-B show the signals detected by some applications of the system measuring a patient in which system 10 has both a motion sensor whose signal is shown in FIG. 8A (the signal being measured in arbitrary units) and an oximeter sensor whose simultaneous signal is shown in FIG. 8B (the signal being measured in arbitrary units). In the time segment preceding 3660 seconds, a high quality signal of the oximeter may be observed, with a pattern that closely follows the heart beat of the patient. Then at around time 3680 seconds a significant motion is detected by the motion sensor and after that the oximeter sensor has fallen from the subject's finger, and the subject turns into prone position and applies force onto the oximeter sensor each time his abdomen moves during the respiratory cycle. Accordingly after about time 3690 seconds, the oximeter sensor detects incorrect results of heart rate and saturation levels generating a potential false alert of low oxygen saturation. In some applications, system 10 identifies the high correlation level between the oximeter sensor signal and the respiratory motion signal after time 3690 (as is also seen in FIGS. 9A-B, which show enlargements of portions of the signals shown in FIGS. 8A and 8B) and the erroneous oximeter alert is accordingly not conveyed to the clinician. For some applications, an alert that is indicative of the oximeter not being correctly positioned is generated in response to the change in the oximetry signal.

As described above, for some applications, system 10 utilizes the combination of the oximeter and the motion sensor to reduce false alarms, since typically a significant change in oxygen saturation is expected to be accompanied by a significant change (increase or decrease) in the patient's respiratory rate and/or heart rate as measured by the motion sensor. For some applications, in order to reduce the number of false alerts in measuring both oxygen saturation and heart rate via the oximeter sensor, correlation with the data from the motion sensor is used. In case a significant drop is identified in the oxygen saturation level, system 10 checks if any significant changes in heart rate or respiratory rate or patterns have been detected in a given time interval prior to the alert, such as, in the previous minute to the previous two hours, e.g., in the previous hour. If such changes (for example, a drop in respiratory rate to below 8 breaths/min) have been detected, system 10 immediately generates an alert and notifies a clinician (as this may be a clear indication of respiratory depression). On the other hand, if the respiratory rates and heart rates have been stable and in normal ranges over that entire time period, system 10 may prevent an alert from being generated. Alternatively or additionally, the system may activate a delay period of 30 seconds to 15 minutes, during which period the oximetry signal is continuously monitored, and if the abnormal readings continue through that period, an alert is generated to notify a clinician. For some applications, this may reduce false alarm rates, while maintaining the risk of missing true patient deteriorations to a low level, since the probability of getting a true saturation alert without any significant change in heart or respiratory rates and patterns is quite low.

For some applications, system 10 monitors (1) the patient's blood oxygen saturation with a photoplethysmograph, and (2) the patient's respiratory motion signal, for example, using a sensor under the patient's mattress, as described hereinabove. For some applications, the combined oxygen saturation and respiratory monitoring is used to detect respiratory diseases or deteriorations other than sleep apnea. Alternatively, the combined oxygen saturation and respiratory monitoring is used to detect sleep apnea.

For some applications, the system determines the correlation between the two signals, and, specifically, analyzes the short term variation of the photoplethysmograph signal (i.e., the variation of the signal over less than 300 seconds, or over less than 30 seconds) and its correlation with the respiratory cycle. For example, the system analyzes the short term variation of the photoplethysmograph signal during inspiration and expiration. This facilitates measurement by the system and/or by the clinician of the expiration versus inspiration times. Changes in the ratio of expiration to inspiration times in the respiratory cycle are in some cases indications of change in respiratory condition, including impending respiratory distress such as an asthma attack.

Furthermore, for some applications of the present invention, short-term changes in oxygen saturation that correlate with the respiratory cycle provide an indication of the patient's respiratory condition. For example, many patients have periods during which the patient's respiration stops for 10-20 seconds (often called hypopnea). For some applications, system 10 identifies such hypopnea events through the measurement of the respiratory motion and analyzes the level of oxygen saturation change in those hypopnea cycles. If such a change is higher than a defined threshold (e.g. 3%), the system indicates that change to the clinician, as indicating that the respiratory system may undergo or be undergoing distress. For some applications, system 10 detects the length of time without breaths that it takes for the patient's oxygen saturation to drop more than a threshold level (generally between 1% and 5%, e.g., between 1.5 and 2.5%, e.g., 2%). Typically, if the subject's respiratory condition is undergoing distress, the threshold drop in oxygen saturation takes place within a given, often shorter length of time. This time level is measured and indicated to a clinician. If the drop in oxygen saturation below the threshold takes place in a shorter length of time than a given threshold time (which may be a set threshold time, or a threshold time that is set based upon the patient's history), an alert is generated to indicate to the clinician that the subject's respiratory condition requires further evaluation. For some applications, system 10 analyzes the variation of the oximetry signal within the respiratory cycle, e.g., averaged over 10 consecutive respiratory cycles (breaths). The standard variation per respiratory cycle is displayed to the clinician.

In some applications, system 10 is configured to detect bed entry and/or exit by subject 12. The system identifies bed entry upon detecting large body movement followed by a signal indicative of continuous motion (e.g., related to respiration or heartbeat), and bed exit upon detecting large body movement followed by a lack of motion signal. For some applications, sensor 30 comprises a single semi-rigid plate, and, coupled thereto, a vibration sensor and two strain gauges that are configured to detect the weight the subject's body applies to sensor 30.

In some applications, system 10 is configured to alert if subject 12 has left the bed and has not returned for a time period that is higher than a specified length of time between 3 minutes and 2 hours, for example 10 minutes. This may be manually or automatically activated for patients for specific times of day, for example during the night. This is useful for supervising patients who may enter or exit the bed independently but may be at risk of falling. The nurse may not want to be alerted every time the patient leaves the bed, but may want to be alerted if the patient left bed and has not returned for 10 minutes, since that could mean that the patient fell and requires assistance or is wandering in the hospital or nursing home with no escort. The nurse may, for example, want this system activated only at night when the nursing team is smaller and the patients are expected to stay in bed practically all the time except for brief bed exits. This ‘long time bed exit alert’ is valuable for reducing the number of alerts and thus “alert fatigue,” while effectively notifying nursing teams of unusual situations that may require interventions.

In some applications of the present invention, system 10 is designed to prevent false alerts that may be generated by an additional person (e.g., a visitor or nurse) who is sitting on the bed in addition to the subject who is being monitored. In some applications, the system comprises a weight sensor that weighs the subject on the bed (as, for example, is installed in several beds manufactured by Stryker Medical of Kalamazoo, Mich. and Hill Rom of Batesville, Ind.). The reading from the weight sensor is communicated through standard communication means to control unit 14. System 10 has a set range of expected weights for the subject (e.g. between 30 and 250 Kg). Before the subject enters the bed, the weight measured is approximately 0. As long as the reading is below the 30 Kg level, the system does not generate any readings. When a weight within the above range is identified, the system automatically initiates measurement. If while measuring the subject a sudden increase in weight is identified of, for example, more than 30 Kg, system 10 recognizes that as an additional person on bed and stops measurement and/or alerts a clinician. This is used to prevent potentially false readings that may be caused due to more than one person being in bed. Alternatively, system 10 includes in some applications an operator interface to indicate to the system when the subject is in bed. The weight measured at that point is logged, and any time that a weight reading that is over 10% above the initial reading is identified, the system stops measurement and/or alerts a clinician.

In addition, in some applications, system 10 uses the weight reading from the weight sensor to identify situations of sudden loss of signal in contactless sensor 30. This loss of signal can be caused by the subject exiting the bed or by a cardiac arrest event. Utilizing the weight reading, system 10 can differentiate between those two scenarios. If the loss of signal is accompanied by a weight drop measured in bed, then the system identifies this as a patient exiting the bed. If such a change in weight is not identified, system 10 identifies this event as a cardiac arrest (for example), and alerts accordingly. In some applications, the bed includes a set of weight sensors that in a combined fashion can calculate the center of mass of the subject (as, for example, are sold by Stryker Medical of Kalamazoo, Mich.). In some applications, system 10 integrates the readings from these weight sensors with a contactless sensor in order to improve the accuracy of detection of a posture change of the subject. A posture change is identified only when the center of mass has shown some movement and the sensor 30 has identified additional features of a posture change as described above. In some applications, the detection of subject entry to and exit from bed, including the identification of an additional subject sitting or lying on the bed, can be identified with a camera coupled to an image processing unit. In some applications, an adaptation of the above described system is implemented for a subject in a chair or wheelchair.

When a clinician evaluates the condition of a patient, in some cases it is useful to combine the current reading of a parameter of the subject's condition with the trend of that parameter over the past few minutes, hours or days. The combination of the current reading and the trend enables an integrated assessment of the subject's current risk level and the need for immediate intervention. For example, a patient whose breathing rate is currently stable at 36 breaths per minute is in very different condition from a patient with the same current breathing rate who until an hour ago had a stable rate of 25 breaths per minute. In some applications of the present invention, system 10 identifies a slow change pattern and is configured with a threshold indicating when the system should generate an alert. The system calculates and outputs the amount of time until the subject will reach the alert threshold if the current slow trend continues. For example, if the system identifies a trend for an increase in breathing rate of 3 breaths/minute every hour, and the current breathing rate is 21 breaths/minute and the threshold is 36 breaths/minute, then the system calculates that the time to alert is 5 hours (5=(36−21)/3) and displays that value of time to alert on the screen. This alert enables the clinician to evaluate the risk level of the current condition based on both the current value and the slow trend. In addition, in some applications, the system outputs a warning if the time to alert is below a threshold value. For example, if the time to alert is less than 2 hours, the system may display a warning message on the screen.

For some applications, a slow-trend pattern of a physiological parameter of the subject is determined based on the three hours or more of sensed data. For example, a pattern may be determined based on three hours or more of sensed heart rate data, respiratory data, and/or motion data. The pattern is compared to previously determined pattern of the physiological data that was based upon sensed data over a similar time frame in the previous 6-24 hours. For example, a pattern based upon data that was sensed between 08:00 and 12:00 may be compared with a pattern based on data that was sensed on the previous evening between 20:00 and 00:00. Alternatively, a pattern based upon data that was sensed between 13:00 and 19:00 may be compared with a pattern based on data that was sensed on the same morning between 03:00 and 09:00. For some applications, in response to changes in a slow-trend pattern of the subject, the sensitivity of the system to short-term changes in parameters of the subject (such as respiration rate or heart rate) is modulated. For example, in response to a pattern based upon data that was sensed between 13:00 and 19:00 as compared with a pattern based on data that was sensed on the same morning between 03:00 and 09:00, indicating that the subject's condition is deteriorating, the sensitivity of the system to short term changes in parameters of the subject (such as respiration rate or heart rate) is modulated.

In some applications of the present invention, system 10 switches between different algorithms for calculating respiratory rates or heart rates between sleep and wake mode, and/or between low activity level and high activity level. For example, for some applications, it is more effective to use a time domain algorithm for calculating respiratory rate when the subject is awake and a frequency domain algorithm when the subject is asleep. Alternatively, the system switches between the different algorithms according to a level of subject activity and/or restlessness. For some applications, upon identifying that a subject is sleeping or in quiet rest, the system activates an early warning mechanism that generates an alert if these is a high risk that the subject will attempt to leave the bed. For example, if the subject is lying quietly in bed and the system suddenly identifies that the subject is moving around in bed continuously for over 30 seconds, the system may generate an alert a clinician that the subject is at high risk of trying to exit the bed. This is useful for preventing subject falls, especially for elderly, demented subjects. For some applications, system 10 builds a baseline of the subject's body movements during sleep and generates an alert upon detecting a movement pattern that is significantly different from baseline, which may indicate that the subject is having trouble sleeping or is transitioning out of sleep. For some applications, the system uses different criteria for generating alerts upon subject movement for different hours of the day. For example, between 2:00 AM and 5:00 AM, a relatively low level of motion in a 30 second interval creates an alert, while at other times of the day the threshold is greater. In some applications, system 10 enables a clinician to designate the subject as a high fall risk patient. For that patient, the system uses more stringent criteria to alert upon motion patterns that may indicate an oncoming fall. For example, the highest risk time period for patient falls for most institutions is the night period (e.g. between 8:00 PM and 5:00 AM). For a patient designated as high risk, the system identifies when the patient is entering rest mode (e.g. low patient motion for over 15 minutes and possibly also reduction of 5% in heart rate vs. the average in the previous 3 hours). Then, after such a rest status is determined, if there is an increase in motion which is above a threshold, an alert is activated to inform the nurse that the patient is not in resting mode any more. For example, if the system identifies large body movements for a period of over 30 seconds, an alert is activated. This may be an indication that the risk of falls has significantly increased and the nurse should attend to the patient as soon as possible. Activating such an alert only at night or only after patient rest is identified helps reduce alerts and accordingly alert fatigue for the clinical team. In some applications, the system is configured to alert upon bed exit of patients who are sedated post surgery for the first few hours while they gradually recover from the effects of sedation. The system has an operator interface that enables the clinician to indicate that a patient is post surgery and to indicate his expected recovery from sedation time. The system generates an alert if the patient attempts to leave bed during that recovery time, e.g. 12 hours, but then automatically turns off the alert feature in order to minimize false alarms. Alternatively, the system turns off the alerts when a motion level indicating full alertness is identified for a set period of time.

For some applications, maximum and/or minimum threshold values for heart rate, respiratory rate and/or oxygen saturation levels of the subject are set on the system. In response to one of the aforementioned parameters decreasing below the minimum threshold, and/or increasing above the maximum threshold, an alert is generated. Typically, in response to the patient maintaining an elevated heart rate, respiration rate, and/or oxygen saturation rate, the maximum threshold for the parameter (and/or one of the other parameters) is raised by a healthcare professional (e.g., a nurse). For some applications, in response to the maximum threshold being raised by the nurse, the system automatically raises the minimum threshold. Thus, if the parameter begins to drop, an alert will be generated by the system sooner than if the minimum threshold had not been raised. Similarly, in response to the minimum threshold being lowered by a nurse, the system automatically lowers the maximum threshold. Alternatively or additionally, in response to the patient's baseline returning to normal, the system narrows the thresholds around the new baseline. For some applications, the system generally adjusts the maximum and minimum thresholds in response to changes in the baseline parameters of the patient. Typically, the system only automatically modifies the thresholds in a manner that is more strict than has been manually input to the system, i.e., the system will automatically raise the minimum threshold and will lower the maximum threshold, but will not lower the minimum threshold or raise the maximum threshold beyond thresholds that have been set by the nurse.

In some applications of the present invention, system 10 helps medical establishments enforce and log the compliance with a pressure ulcer prevention protocol. For example, in many hospitals, the protocol for preventing pressure ulcers in patients who are considered at high risk for such ulcers is to have the patients turned over once every 2 hours. In some applications, system 10 comprises a user interface that enables a clinician (e.g. physician or head nurse) to indicate the required protocol to prevent pressure ulcers, e.g., the maximal amount of time allowed between posture changes due to the patient turning or the patient being turned. The system's user interface 24 then displays a counter counting down the time till the next required posture change of the patient, according to the protocol. If that counter reaches zero an alarm is activated. If the system identifies a posture change, the counter is reset to the original value (e.g. 2 hours) and initiates the countdown again.

In some applications, system 10 includes a double layer of protection to prevent a false detection of a patient being turned. In order to make the identification of a posture change and to reset the counter, it requires both a posture change to be detected via the sensor and control unit and the clinician to make an input via the user interface that he/she actually turned the patient. So, in order to reset the counter, system 10 requires the clinician input and sensor input regarding posture change to coincide within a set period of time (e.g., 10 to 300 seconds, typically 60 seconds). Thus, when the nurse approaches the pressure ulcer risk patient to turn him, she presses the appropriate button on the user interface and then turns the patient. The system identifies the turn through its sensor and accepts the input through the user interface; if they both coincide within (for example) 60 seconds, then the counter is reset. In some applications, the system also logs every such event to help document patient care and reduce hospital liability. In some applications, the detection of posture change is implemented without contacting the subject's body, via a sensor under the mattress or a camera.

In some applications, system 10 combines two sensing elements: a camera and a motion sensor. The signal from the two sensors is correlated in order to reduce artifacts. For each sensor, a confidence value is calculated for each reading, and the source with the higher confidence level is selected. Alternatively, a clinical parameter (e.g. heart rate) is calculated independently from the signal of each sensor. If the two readings are similar within a set range, the readings are allowed, displayed, and logged. If relevant, alerts are created. If the signals are different, they are rejected.

In some applications of the present invention, system 10 is integrated into a communication system wherein information for multiple systems 10 is accumulated and presented in a central display station located, for example, in the hospital's nurse station, or at a call center, for patients at home. In such environments, there are often multiple nurses each assigned to take care of a group of one or more patients within the unit or region. It may useful to allow grouping patients assigned to a specific nurse in a convenient, easy-to-view way that can be understood with a quick glimpse as a clinician walks by a display. In some applications, each nurse is assigned a color at the beginning of his/her shift. All patients assigned to that nurse are then automatically or manually assigned to the same color. That information is entered into the central display station through an input means (for example, utilizing a keyboard or touchscreen), or received automatically (for example, from a hospital's computerized ADT (Admit Discharge Transfer) system). The central display groups patients by their color coding, so the nurse can view his/her group while walking by the display and easily see whether any of his patients require immediate attention. Furthermore, in some applications, the nurse also gets assigned a mobile phone or pager for his/her shift. This phone is marked with the same color assigned to the nurse, and accordingly the nurse gets the alerts related only to the patients assigned to that nurse. In another embodiment, the vital signs information about each patient can be presented in matrix form, so each column may represent a different nurse.

For some applications, system 10 is utilized to monitor ventilated patients, for example, as described in US 2008/0275349 to Halperin et al., which is incorporated herein by reference. System 10 utilizes a sensor under the patient's mattress, which is optionally contact-less, in order to continuously monitor the mechanical motion signal of a ventilated patient and, upon detecting a change in the motion signal, to alter the ventilation status of the patient, e.g., by changing the physical position of the ventilation tube, taking out the tube, and/or changing the ventilation system parameters.

For some applications, system 10 includes a piezoelectric sensor installed on a semi-rigid plate which is placed on the bed frame under the mattress, to monitor the patient's breathing and heart parameters, as described hereinabove. The plate is installed in the top section of the bed where usually the patient's head and chest is located. The sensor plate also has an accelerometer installed thereon, to facilitate measurement of the tilt angle of the plate.

In many beds, the top section of the bed may optionally be angled upwardly. In general, orienting the top section of the bed at an angle that is greater than a given angle with respect to a lower section of the bed has been shown in clinical trials to be helpful in preventing various respiratory diseases, and in helping patients who have respiratory diseases to recuperate faster. Specifically, for patients who are ventilated, some clinical work has shown that orienting the top section of the bed at an angle that is greater than 30 degrees (e.g., greater than 45 degrees) with respect to a lower section of the bed significantly reduces the incidence of ventilator-associated pneumonia, which is a significant concern for ventilated patients.

For some applications, system 10 continuously logs, and optionally displays, the angle of the top section of the bed, as detected by the accelerometer. For some applications, system 10 generates an alert if the angle is below a set threshold (e.g., below 30 degrees), or alternatively if an angle below that threshold has been maintained for a time period that is greater than a given threshold (e.g., if the angle has been set at an angle smaller than 30 degrees for more than 8 consecutive hours, or if the angle has been below 30 degrees for more than 12 hours during the last 24 hour period).

For some applications, in order to prevent unnecessary alerts and reduce clinician alarm fatigue, system 10 uses the respiratory or cardiac related motion detected through the piezoelectric sensor in order to identify that a patient is in bed, and the timer that counts the amount of time the patient is at a low angle is only activated if the patient is actually in bed.

For some applications, the piezoelectric sensor is used to detect when a patient is on a ventilator by identifying a characteristic of a signal that is generated in response to a parameter of the patient when a ventilator is active. For example, when a ventilator is active, the variability of the breathing motion signal between consecutive breaths is significantly smaller than with non-ventilator assisted breaths or when the ventilator is active. Therefore, a variability of the breathing motion signal between consecutive breaths may be detected in order to detect when the ventilator is active. Or alternatively, identifying a signal having a frequency that is the frequency at which electricity is provided (e.g., 50 or 60 Hz), as described herein. Alternatively, an additional vibration sensor may be placed on the ventilator itself, or a digital communication signal may be received from the ventilator into system 10 to indicate that the ventilator has been activated. For some applications, the system continuously logs, and optionally displays, the activation times of the ventilator, as well as the head-of-bed angle. Alternatively or additionally, if the angle of the top section of the bed is below a threshold angle (e.g. 45 degrees) for an extended period of time, or for more than a threshold percentage of the time that the patient is on an active ventilator, an alert is generated. For some applications, the angle of the top section of the bed and the ventilation information are continuously displayed for effective management purposes of the nursing staff.

For some applications, system 10 has a barcode reader integrated therewith, such that the system is able to automatically read a barcode that identifies the patient, a nurse (who performs changes or responses to the system), and/or medication that is administered to the patient. For some applications, administration of medication to the patient is logged by the system, and the system detects changes in parameters of the patient (e.g., the patient's heart rate, respiratory rate, and/or oxygen saturation) that may be associated with the administration of the medication to the patient. For some applications, an alert is generated in response thereto.

For some applications, system 10 is used to monitor the patient both in the patient's home and an in a hospital environment. When the patient is in the hospital and parameters of the patient (e.g., the patient's heart rate, respiratory rate, oxygen saturation, and/or sleep pattern) are detected that indicate that the patient may be ready to be discharged from hospital, the system generates a notification. In response to the notification, a clinician evaluates whether the patient may be discharged. Alternatively or additionally, when the patient is in the home and parameters of the patient (e.g., the patient's heart rate, respiratory rate, oxygen saturation, and/or sleep pattern) are detected that show a similarity to parameters of the patient that preceded previous hospitalizations of the patient, an alert is generated. In response thereto, a clinician determines whether to hospitalize the patient.

It is noted that the terms “patient” and “subject” are used interchangeably throughout specification and claims of the present application and that any instance in which the term “patient” is used, it could be substituted by the term “subject,” and vice versa.

Techniques described herein may be practiced in combination with techniques described in one or more of the following patents and patent applications, which are incorporated herein by reference. In some applications, techniques and apparatus described in one or more of the following applications are combined with techniques and apparatus described herein:
U.S. Provisional Patent Application 61/052,395 filed May 12, 2008;
U.S. Provisional Patent Application 61/054,754 filed May 20, 2008;
U.S. Provisional Patent Application 60/674,382 filed Apr. 25, 2005;
U.S. Provisional Patent Application 60/692,105 filed Jun. 21, 2005;
U.S. Provisional Patent Application 60/731,934 filed Nov. 1, 2005;
U.S. Provisional Patent Application 60/784,799 filed Mar. 23, 2006;
U.S. Provisional Patent Application 60/843,672 filed Sep. 12, 2006;
U.S. Provisional Patent Application 60/924,459, filed May 16, 2007;
U.S. Provisional Patent Application 60/924,181, filed May 2, 2007;
U.S. Provisional Patent Application 60/935,194, filed Jul. 31, 2007;
U.S. Provisional Patent Application 60/981,525, filed Oct. 22, 2007;
U.S. Provisional Patent Application 60/983,945, filed Oct. 31, 2007;
U.S. Provisional Patent Application 60/989,942, filed Nov. 25, 2007;
U.S. Provisional Patent Application 61/028,551, filed Feb. 14, 2008;
U.S. Provisional Patent Application 61/034,165, filed Mar. 6, 2008;
U.S. Provisional Application 61/082,510, filed Jul. 22, 2008;
U.S. Provisional Application 61/103,276, filed Oct. 7, 2008,
U.S. Provisional Application 61/141,677, filed Dec. 31, 2008;
U.S. Provisional Application 61/144,743 filed Jan. 15, 2009;
U.S. patent application Ser. No. 11/197,786, filed Aug. 3, 2005, which issued as U.S. Pat. No. 7,314,451;
U.S. patent application Ser. No. 11/782,750, filed Jul. 25, 2007, which published as US 2008/0269625;
U.S. patent application Ser. No. 11/446,281, filed Jun. 2, 2006, which published as US 2006/0224076;
U.S. patent application Ser. No. 11/755,066, filed May 30, 2007, which published as US 2008/0114260;
U.S. patent application Ser. No. 12/113,680 filed May 1, 2008, which published as US 2008/0275349;
U.S. patent application Ser. No. 11/048,100, filed Jan. 31, 2005, which issued as U.S. Pat. No. 7,077,810;
International Patent Application PCT/IL2005/000113, which published as WO 2005/074361;
International Patent Application PCT/IL2006/000727, which published as WO 2006/137067;
International Patent Application PCT/IL2006/002998, which published as WO 2007/052108; and
International Patent Application PCT/IL2009/000473, which published as WO 2009/138976.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus comprising:
a motion sensor configured to sense motion of a subject and to generate a motion signal in response thereto;
an oximetry sensor configured to measure oxygen saturation of the subject and to generate an oximetry signal in response thereto; and
a control unit configured to analyze the sensed motion and the sensed oximetry signal, and filter out false alerts relating to a condition of the subject generated by the oximetry signal, based on correlation between the oximetry signal and an aspect of the motion signal.

2. The apparatus according to claim 1, wherein the control unit is configured to filter out the false alerts based on correlation between the oximetry signal and an aspect of the motion signal relating to a respiratory cycle of the subject.

3. The apparatus according to claim 1, wherein the motion sensor is configured to sense motion of the subject without contacting or viewing the subject or clothes the subject is wearing.

4. The apparatus according to claim 1, wherein the motion sensor comprises a camera configured to acquire images of the subject, and wherein the control unit is configured to reduce an identifiability of portions of the subject's body in the images, by applying a contour detection algorithm to the images.

5.-36. (canceled)

37. Apparatus comprising:
a respiratory sensor configured to sense respiratory motion of a subject in a bed, and to generate a respiratory signal in response thereto;
an oximetry sensor configured to detect oxygen saturation of a subject, and to generate an oxygenation signal in response thereto; and
a control unit configured to:
determine a correlation between the respiratory signal and the oxygenation signal, and generate an alert that is indicative of an abnormal respiratory condition of the subject, in response to detecting a change in the correlation.

38. The apparatus according to claim 37, wherein the control unit is configured to generate the alert in response to detecting a decrease in the correlation between the respiratory signal and the oxygenation signal.

39. The apparatus according to claim 37, wherein the respiratory sensor comprises a contact-less sensor that is configured to detect the subject's respiratory motion without contacting or viewing the subject or clothes that the subject is wearing.

40. The apparatus according to claim 37, wherein the control unit is configured to generate the alert in response to:
determining that the subject has stopped breathing, in response to the respiratory signal, and
determining that, since the subject stopped breathing, the time it takes the oxygen saturation level to drop by a threshold amount is less than a threshold time period.

41.-60. (canceled)
